(19) United States
(12) Patent Application Publication
(10) Pub. No.: US 2025/0160736 A1
(43) Pub. Date: May 22, 2025

(54) COOPERATIVE LEARNING FOR PERSONALIZED CONTEXT-AWARE PAIN ASSESSMENT

(71) Applicant: UNIVERSITY OF SOUTH FLORIDA, Tampa, FL (US)

(72) Inventors: Shaun CANAVAN, Tampa, FL (US); Md Taufeeq UDDIN, Tampa, FL (US); Ghadh ALZAMZMI, Tampa, FL (US)

(21) Appl. No.: 18/954,450

(22) Filed: Nov. 20, 2024

Related U.S. Application Data
(60) Provisional application No. 63/600,774, filed on Nov. 20, 2023.

(51) Int. Cl. A61B 5/00

(52) U.S. Cl. CPC A61B 5/4824; A61B 5/7267

(57) ABSTRACT

Technology for cooperative learning pain assessment may include a sensed pain classifier to determine a sensed context and a pain assessment classifier to determine an inferred pain score. Disclosed technology may determine an uncertainty score based on the sensed context and the inferred pain score. Responsive to the uncertainty score meeting a first threshold condition, a manual label for the subject data may be used to update the sensed pain classifier or the pain assessment classifier. Responsive to the uncertainty score meeting a second threshold condition, a generated label for the subject data may be used to update the sensed pain classifier or the pain assessment classifier. The inferred pain score may be provided as an assessed pain result for the subject.

### CROSS-REFERENCE TO RELATED APPLICATIONS

This application claims the benefit of U.S. Provisional Patent Application Ser. No. 63/600,774, filed Nov. 20, 2023, the disclosure of which is hereby incorporated by reference in its entirety, including all figures, tables, appendices, and drawings.

### STATEMENT REGARDING FEDERALLY SPONSORED RESEARCH OR DEVELOPMENT

This invention was made with government support under Cooperative Agreement Number W911NF-22-2-0001 awarded by the Army Research Office DEVCOM Analysis Center. The government has certain rights in the invention.

### TECHNICAL FIELD

Various embodiments and implementations described herein relate generally to systems and methods for effective computing. More specifically, embodiments and implementations hereof may involve cooperative learning for pain assessment.

### BACKGROUND

Affective computing applications have emerged to enhance healthcare. This includes intelligent monitoring of physiological changes and sedation states as well as assessment of affective states such as pain. Although affective computing applications achieved promising performance, they still suffer from performance generalization due to the lack of relatively large and annotated datasets. This scarcity is magnified in healthcare applications due to patient privacy and data protection laws, and the high cost of acquiring medical expert annotations. To tackle the data scarcity issue, machine learning approaches such as semi-supervised learning, and active learning have been proposed.

Active learning tackles data scarcity by interactively picking, from a pool of unlabeled data, a set of influential examples for labeling by human experts. This interactive process of learning has gained attention in the affective computing community as it mitigates the issues of data scarcity and reduces the labeling cost. Examples of affective computing applications that use active learning approaches include online audio-visual emotion recognition and acoustic emotion recognition.

Several methods have been proposed to automatically assess pain based on the analysis of a single pain modality such as facial expression, sound signals, body movement signals, and physiological signals. Other methods investigated combining different pain modalities (multimodal) to obtain robust and reliable assessment during missing data. Although several unimodal and multimodal approaches are proposed, most of these methods are trained to learn a function that maps the input data to an output label based on labeled input-output data pairs. In the medical domain, obtaining relatively large and well-annotated pain datasets is expensive due to patient privacy constraints and the tedious annotation process.

### SUMMARY

Some aspects of the present technology include pain assessment systems, method, and apparatuses. For example, a system may include a wearable sensor to obtain sensor data for a subject, a context input to obtain pain context data for the subject, a processor, and a non-transitory computer readable medium storing instructions executable by the processor. For instance, the instructions may be executable by the processor to apply a sensed pain classifier to the sensor data to determine a sensed context, apply a pain assessment classifier to the pain context data and the sensed context to determine an inferred pain score, determine an uncertainty score based on the sensed context and the inferred pain score, and output the inferred pain score for the subject. In some examples, the instructions may be executable to, responsive to the uncertainty score meeting a first threshold condition, request a manual label for the sensor data and update the sensed pain classifier or the pain assessment classifier based on the manual label. In some examples, the instructions may be executable to, responsive to the uncertainty score meeting a second threshold condition, generate a generated label for the sensor data and update the sensed pain classifier or the pain assessment classifier based on the generated label.

### BRIEF DESCRIPTION OF THE DRAWINGS

FIG. 1 shows an example of a system for cooperative learning for personalized context-aware pain assessment.

FIG. 2 shows a further example system for cooperative learning for personalized context-aware pain.

FIG. 3 is a flow diagram illustrating an example process for pain assessment.

FIG. 4 is a flow diagram illustrating an example process for generating pain assessment classifiers.

FIGS. 5A and 5B illustrate a summary of a dataset used for various experiments and simulations.

FIGS. 6A and 6B illustrate simulation results with respect to various pain assessment modalities.

FIGS. 7A and 7B illustrate results of a simulated cold start scenario of training existing classifiers with new users.

FIGS. 8A and 8B illustrate example simulation results with respect to varying a first system parameter.

FIGS. 9A and 9B illustrate example simulation results with respect to varying a second system parameter.

FIGS. 10A and 10B illustrate example simulation results with respect to varying a third system parameter.

FIGS. 11A and 11B (MCC) illustrate example simulation results with respect to varying a fourth system parameter.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

Aspects of the present technology may provide a cooperative machine learning system may provide a framework that allows the model to perform most of the labeling and collaborate with the user (e.g., patient, healthcare provider, parent, etc.) to obtain labels during uncertainty. This can mitigate annotation scarcity, dramatically reduce labeling cost, and gradually increase model performance. It may also enable responsible communication between model and user, which allows customization and improved the user's experience and trust. Internal and external contexts, and individual differences may be integrated into pain assessment models. Pain assessment may be performed as a proactive task representing the evolving nature of context and human behavior over time.

Aspects of the present technology may improve the pain assessment performance with the incorporation of context and personalization while being resource efficient and interactive. As discussed below, experimental results have demonstrated competitive performance with a limited number of human-labeled examples, with statistically significant performance improvement using our framework with personalization in the cold start scenario.

It is known that human behavior evolves over time and that human health conditions are subject to change. For example, the patient's status of chronic lower back (CLB) pain may change from no-pain to pain or recovered from pain due to proper intervention and rehabilitation. This information is relevant and useful for predicting the pain experienced throughout the treatment course for a given patient as it provides personalization and contextualization. However, the evolving nature of human behaviors and health conditions requires the development of pain assessment models that are robust to such changes and can be adjusted throughout the lifespan of a pain assessment model. Some aspects of the proposed technology leverage cooperation between humans and a pain assessment model to collect new annotated examples for adapting to changes by quantifying the quality of the prediction using uncertainty values, such as a model entropy quantification method.

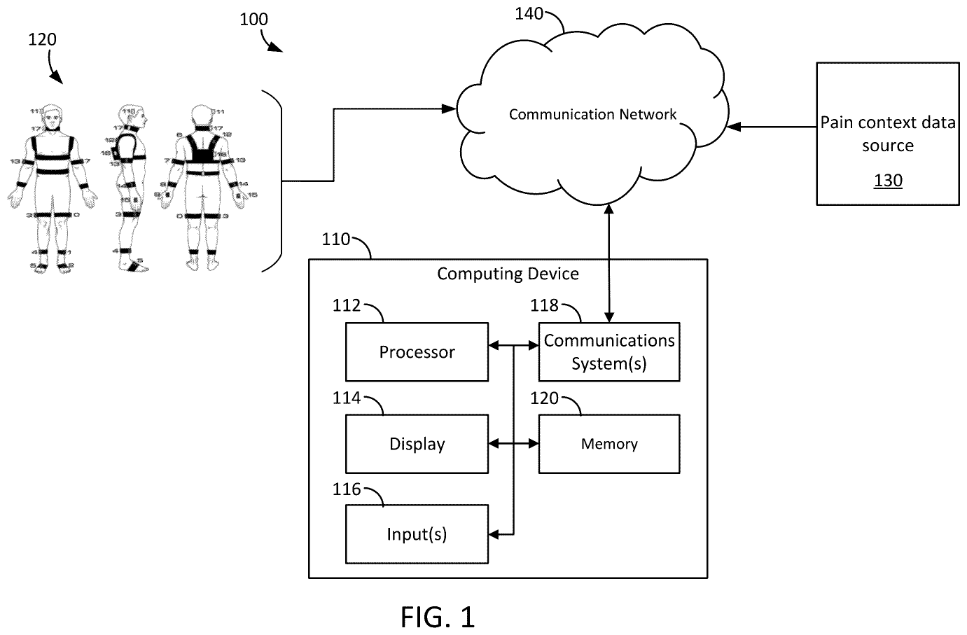

FIG. 1 shows an example 100 of a system for cooperative learning for personalized context-aware pain assessment in accordance with some embodiments of the disclosed subject matter. As shown in FIG. 1, a computing device 110 can receive subject sensor data 120 from one or more sensors. For example, sensor data 120 may comprise data received from wearable devices worn by a subject. For instance, sensor data 120 may include sensing data such as body movement, posture information, heart rate, breathing rate, and muscle movement (e.g., EMG) data. As a further example, sensor data 120 may include externally recorded data (e.g., video or audio) obtained while from a subject. In some implementations, sensor data 120 may comprise data obtained from a subject while performing known pain-inducing movements (e.g., bending, one-leg-stand, reach-forward, sit-to-stand, stand-to-sit, etc.), data obtained from a subject while performing known movements that do not induce pain (e.g., sitting still, standing still, walking, self-preparation, etc.). In further implementations, sensor data 120 may be data received while a subject is performing various motions without a priori information about their pain-inducing properties (e.g., data recorded while a subject performs typical day-to-day movements, etc.).

As further shown in FIG. 1, a computing device 110 can receive pain context data from a pain context data source 130. In some examples, pain context data source 130 may comprise a wearable sensor or other sensor data source, such as, for example body movement patterns captured from wearable devices. As another example, pain context data source 130 may comprise a data store (e.g., database or other record structure) storing contextual data for a subject's pain. Examples of contextual information that are known prior to pain assessment include medications and diagnostic test results, which may be available in the patient's health record. Examples of contextual information that can be estimated from the sensing data include body movement patterns captured from wearable devices.

For example, pain context data may include medical records/diagnoses (e.g., if a subject has had a prior injury, been previously diagnosed with a pain-related condition such as a chronic lower back pain (CLB), etc.). As another example, pain context data may include demographic data, such as data regarding the subject's age, gender, occupation, etc. As a further example, pain context data can include subject-specific contextual data, such as a subject's self-reported pain, activities, etc. For instance, pain context data for an athlete undergoing pain assessment might comprise information regarding the athlete's particular sport, activities within this particular sport (e.g., position played, activities which cause pain, etc.). As another example, pain context data may comprise results of previous pain assessments, ground truth labels for training samples, etc. As a further example, subject sensor data 120/pain context data 130 may be received from a database of labeled training samples, such as from a publicly available dataset, previous subject data, data obtained from other subjects, etc.

In some examples, the computing device 110 can receive the data over a communication network 140. In some examples, the communication network 140 can be any suitable communication network or combination of communication networks. For example, the communication network 140 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 140 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, the computing device 110 can be any suitable computing device or combination of devices, such as a desktop computer, a laptop computer, a smartphone, a tablet computer, a wearable computer, a server computer, a camera, a virtual machine being executed by a physical computing device, etc. For example, computing device 110 may comprise a wearable computer (e.g., a smartwatch, an athletic or military sensor vest, a mobile phone, etc.), a mobile device connected to a wearable computer, a clinical computer/server, etc.

In some examples, the computing device 110 can execute various classifiers to determined corresponding pain scores. For example, computing device 110 can execute a sensed pain classifier to determine sensed contexts from the sensor data. For example, the sensed pain classifier may be applied known sensing data such as body and muscle movement to encode unknown contextual (EUC) information, which may provide an estimate of an unknown pain context in the form of class probabilities. For example, the sensed pain classifier may provide an inferred pain score based on sensor data without associated contextual data (e.g., a class prediction for a scaled pain value, such as an integer from 1-3, 1-10, etc. (e.g., where 3, 10, etc. indicate a highest pain level, and 1 indicates a lowest pain level, etc.)). As another example, the sensed pain classifier may output other sensed contexts, such as, for example a latent vector or other learned representation of the data.

As another example, the computing device 110 can execute a pain assessment classifier to determine an inferred pain score from the pain context data and the sensed context. For example, the pain assessment classifier may be applied to the output of the sensed pain classifier, further associated context data, the subject sensor data, or combinations thereof. In some examples, the inferred pain score may represent a target/output assessed pain score. In some cases, the pain assessment classifier may provide an output similar to the sensed pain classifier. For instance, the pain assessment classifier may output a class probability vector, latent vector, etc.

In some implementations, the sensed pain classifier and the pain assessment classifier may have similar architectures. For instance, the classifiers may comprise random forest (RF) classifiers. For example, in some cases, sensor/context data may be tabular data, which is well-suited to tree-based models. As another example, RF may provide a reasonably rapid computation, out-of-bag estimation, generalization with less parameter tuning, etc. As a further example, RF may handle cases of scarce and skewed (outliers) training examples. In further examples, the classifiers may comprise any suitable classifier such as neural networks, support vector machines, decision classifiers, etc. In further implementations, the sensed pain classifier and pain assessment classifiers may have different architectures. For instance, the sensed pain classifier might be an RF with the pain assessment classifier comprising a neural network, etc. Although the system described here references two pain classifiers (sensed pain and pain assessment), alternative realizations of the system could be in the form of a sequence of additional classifiers or a hierarchy of classifiers for pain assessment.

In further examples, the computing device 110 can include a processor 112, a display 114, one or more inputs 116, one or more communication systems 118, and/or memory 120. In some implementations, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), neuromorphic processor, accelerator, etc. In some implementations, the display 114 can include any suitable display devices, such as a computer monitor, a touchscreen, a television, an infotainment screen, etc. In some implementations, the input(s) 116 can include any suitable input devices and/or sensors that can be used to receive user input, such as a keyboard, a mouse, a touchscreen, a microphone, etc. In some implementations, input(s) 116 may include sensors, such video recording device, wearable sensors, electromyogram (EMG) sensors, accelerometers, pulsometer, inertial measurement units (IMUs), oximeters, etc.

In further examples, the communications system(s) 118 can include any suitable hardware, firmware, and/or software for communicating information over communication network 140 and/or any other suitable communication networks. For example, the communications system(s) 118 can include one or more transceivers, one or more communication chips and/or chip sets, etc. In a more particular example, the communications system(s) 118 can include hardware, firmware and/or software that can be used to establish a Wi-Fi connection, a Bluetooth connection, a cellular connection, an Ethernet connection, a USB connection, etc.

In further examples, the memory 120 can include any suitable storage device or devices that can be used to store image data, instructions, values, AI models, etc., that can be used, for example, by the processor 112 to perform pain assessments to present content using display 114, to receive image sources via communications system(s) 118, etc. The memory 120 can include any suitable non-transitory computer readable medium, such as volatile memory, non-volatile memory, storage, or any suitable combination thereof. For example, memory 310 can include random access memory (RAM), read-only memory (ROM), electronically erasable programmable read-only memory (EEPROM), one or more flash drives, one or more hard disks, one or more solid state drives, one or more optical drives, connectivity of a field programmable gate array (FPGA), etc. In some embodiments, the memory 120 can have encoded thereon a computer program for controlling operation of computing device 110. For example, in such embodiments, the processor 112 can execute at least a portion of the computer program to perform one or more data processing and identification tasks described herein and/or to train/run classifiers based on sensory data 120 and context data 130 described herein, present content to the display 114, transmit/receive information via the communications system(s) 118, etc. As another example, processor 112 can execute at least a portion of system 200, processes 300, 400, or any other operation described herein.

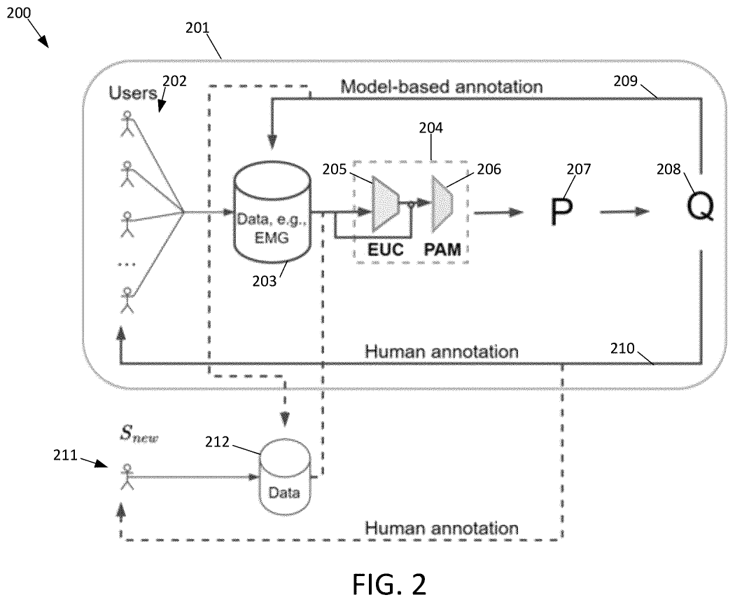

FIG. 2 illustrates an example system 200 for cooperative learning for personalized context-aware pain assessment in accordance with some embodiments of the disclosed subject matter. For example, system 200 may be implemented via a computing system as described with respect to FIG. 1. The cooperative machine learning system 200 may provide a framework that allows the model to perform most of the labeling and collaborate with the user (e.g., patient, healthcare provider, parent, etc.) to obtain labels during uncertainty. This can mitigate annotation scarcity, dramatically reduce labeling cost, and gradually increase model performance. It may also enable responsible communication between model and user, which allows customization and improved the user's experience and trust.

In this example, pain assessment classifiers 204 are trained on initial seed data 203 based on data subject data 202. For instance, subject data 202 and seed data 203 may comprise user data obtained via a labeled training dataset (e.g., a publicly available dataset), from prior pain assessment system subjects, seed data 203 obtained from multiple measurements/assessments of a single user, etc. In various implementations, seed data 203 may comprise sensed pain data and pain context data as described above.

Continuing the example, the seed data 203 may be evaluated via one or more classifiers 204. For instance, a sensed pain classifier 205 may be applied to data 203 to output a sensed context. The output of sensed pain classifier 205 may provide an inferred/encoded unknown context derived from the sensor data. In this example, the output of the sensed pain classifier 205 and data from dataset 203 may be combined and input to a pain assessment classifier 206. The pain assessment classifier 206 may output an inferred pain score 207, which may be provided to the subject, a clinician, a coach, etc.

In some examples, an uncertainty score 208 may be determined based on the outputs of classifiers 204. For instance, the uncertainty score 208 may provide a value indicative of a quality of the assessed pain prediction output by the pain assessment classifier 206. For example, the uncertainty score 208 may be calculated based on combined uncertainty measurements of the sensed pain classifier 205 and the pain assessment classifier 206. As a particular example, the uncertainty score 208 may be determined as:

$$Q = ( 1 - λ ) {E}_{euc} + λ {E}_{pam} , \qquad ( 1 )$$

where E_{euc} represents the entropy of the output of the sensed pain classifier 205, Eₚₐₘ represents the entropy of the output of the pain assessment classifier 206, and λ represents a weighting parameter. For example, in an implementation where classifiers 205, 206 output class probabilities, the entropy E may be determined as:

$$E = - {\sum }_{c ∈ C} {p}_{c} * log ( {p}_{c} ) ,$$

where p_{c} is the probability of each class c in C. In some examples, λ may be system hyperparameter. For example, λ may be set by a system operator, a user, may be preprogrammed, etc. In other examples, λ may comprise a learned parameter which may be learned/updated during training or system operation. In the above example, a value of λ=0.5 weights the entropy of the sensed pain classifier 205 and pain assessment classifier 206 equally, a value of λ>0.5 weights the pain assessment classifier 206 as more influential to the uncertainty score, and a value of λ>0.5 weights the sensed pain classifier 205 as more influential. In such examples, λ may be any weighting parameter bounded between [0,1], such as for example, a weight within the range [0.2, 0.8], [0.3, 0.7], etc. In some examples, λ remains fixed for a particular pain assessment model, while in others λ may be adjusted during model operation.

In some examples, the uncertainty score 208 is used to guide a cooperative learning process where classifiers 204 are updated based on a cooperation between model based data labeling 209 and manual data labeling 210. For example, the label may be an inferred pain score P 207 that is appended to the data sample corresponding to the score P 207. For example, system 201 may automatically generate a label 209 for a data sample if the uncertainty score is sufficiently low (e.g., if the output 207 is sufficiently certain). For instance, if the uncertainty score is below a threshold value or is a member of a low uncertainty group, etc. In some examples, system 201 may transmit 210 a request for a manual label for a data sample if its uncertainty score is sufficiently high. For instance, if the uncertainty score exceeds a second threshold value, or is a member of a high uncertainty group, etc.

In some implementations, an existing trained model 201 may be instantiated for pain assessment of a new subject 211. In this example, data 212 such as sensor data and pain context data for the new subject 211 may be combined with data 203 and provided to classifiers 204 to generate an inferred pain score 207 and uncertainty score 208. The uncertainty score 208 may be used to determine whether the new data will be labeled (e.g., either manually or automatically), thus updating the existing trained model 201 with new user data 212.

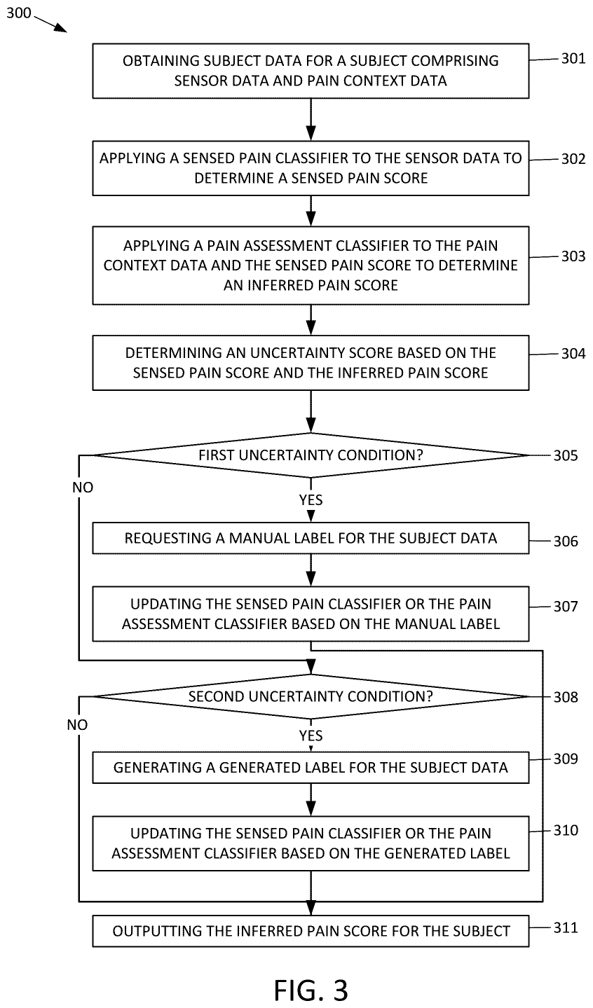

FIG. 3 is a flow diagram illustrating an example process 300 pain assessment in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., computing device 110) in connection with FIG. 1 can be used to perform the example process 300. However, it should be appreciated that any suitable apparatus or means for carrying out the operations or features described below may perform the process 300. The process 300 is generally directed to a runtime stage using one or more trained classifiers. As discussed below, during the runtime stage, the trained classifiers may be updated (e.g., trained further). Generating initial trained classifiers is described in connection with FIG. 3. In various implementations, the process 300 can be used for any pain assessment scenario, such as athletic pain assessment, postoperative pain assessment, chronic pain assessment, at-home healthcare pain assessment, battlefield pain assessment, rehabilitation pain assessment, etc. However, the process 300 can be used for any other suitable purposes, such as pain prediction, etc.

In some examples, process 300 may include step 301, which may include obtaining subject data for a subject comprising sensor data and pain context data. For example, step 301 may include collecting sensor data (e.g., body movement, muscle movement, other physiological data) using wearable devices or other devices (e.g., video of a subject performing various motions) as discussed above with respect to sensor data 120. Step 301 may further include obtaining pain context data as discussed with respect to pain context data source 130. For example, step 301 may include obtaining sensor data annotated with labels or otherwise associated with pain context data. For instance, step 301 may include obtaining tabular data comprising samples with associated context. For example, step 301 may comprise obtaining various feature vectors indicative of sensed or contextual data. As a particular example, step 301 may include receiving a body sensor feature vector (e.g., a representation of angular velocity, posture angle, etc.) along with context variables (e.g., binary variables indicating a certain status, such as CLB status, or other values, such as age). As another example, context data may include variable derived from sensor data or other information, such as a subject performing a pain inducing or non-pain inducing activity, etc.

In some examples, process 300 may include step 302, which may include applying a sensed pain classifier to the sensor data to determine a sensed context. For instance, process 300 may be performed during a classifier training process, a cold-start process, or an operational pain assessment process (e.g., as discussed below with respect to FIG. 400). For example, step 302 may include inputting sensor data obtained in step 301 into a random forest classifier to determine a sensed context. For example, the sensed context may be a pain level classification (e.g., mild, severe), a probability vector (e.g., probabilities of different predicted pain levels, a latent feature vector, etc. In some examples, the sensed context may be a probability of one or more contextual variables (e.g., a probability that a subject was performing a pain-inducing activity), etc. As a particular example, step 302 may comprise applying a first random forest classifier to the sensor data.

In some examples, process 300 may include step 303, which may include applying a pain assessment classifier to the pain context data and the sensed context to determine an inferred pain score. For example, the pain assessment classifier may generate an inferred pain score based on unknown contextual information provided by the sensed pain classifier, the sensed data from the subject, etc. For example, the inferred pain score may be a pain level classification (e.g., mild, severe), a probability vector (e.g., probabilities of different predicted pain levels, a latent feature vector, etc. As a particular example, step 303 may comprise applying a second random forest classifier to the sensor data.

In some examples, process 300 may include step 304, which may include determining an uncertainty score based on the sensed context and the inferred pain score. For example, step 304 may include computing an indication of uncertainty of predictions/classification made by the sensed pain classifier and the pain assessment classifier. For instance, the uncertainty score may comprise a linear combination of prediction entropies for the sample under evaluation as discussed above with respect to eq. (1).

In some examples, process 300 may include step 305, which may include determining whether the uncertainty score meets a first uncertainty condition. If the uncertainty condition is met, then process 300 may include step 306 of requesting a manual label for the subject data. For example, in one example, steps 301-303 may be performed multiple times to determine a pooled dataset. In this example, after computing the uncertainty score on the pooled dataset using eq. (1), the examples in dataset may be ranked from most uncertain to certain (e.g., high Q indicates high uncertainty) by sorting examples in descending order using the Q associated with each example. In this example, the top k examples (i.e., most uncertain) from the ranked dataset may be sent for manual labeling. For instance, they may be sent to the subject for their feedback regarding sensed pain associated with the data (e.g., how painful the activity corresponding to the sample was). As another example, data may be sent to a practitioner or other user for their feedback (e.g., for a parent or caregiver evaluating a non-verbal subject), etc. In some examples, k may be a configurable system parameter. For instance, k may determine an amount of time required for a user to interface with the system (e.g., evaluating 5 samples will generally take less time than evaluating 10 samples, etc. In some examples, k may be any number between, for example, 1-100 or more, such as, for example 5, 10, 15, 25, 50 samples, etc. The examples described below provide further examples how k may be established. In some examples, k may be a learnable parameter or a dynamic parameter, etc. For instance, k may reduce as the size of a subject's data pool increases.

In other examples, steps 305 and 306 may be performed based on other factors. For instance, samples exceeding a threshold uncertainty value may be sent for manual labeling. For instance, during a cold-start scenario a subject may perform movements under supervision from a practitioner. In this example, samples and uncertainty values may be obtained in real-time and a subject may be asked to label samples as they are obtained. For instance, a subject may perform a session (e.g., a few minutes, tens of minutes, an hour, etc.). During the session, a subject may perform a motion which is then evaluated by the classifiers to determine an uncertainty score and pain assessment. For instance, the uncertainty score and assessment may be displayed to the user on a graphical user interface (GUI). If the uncertainty score meets a threshold condition the user may be asked to label the sample. The model may then be updated based on a label and a next sample may be taken.

In some examples, process 300 may include step 307, which may include updating the sensed pain classifier or the pain assessment classifier based on a manual label obtained in step 306. For instance, the sensed pain classifier and the pain assessment classifier may be retrained after appending the newly labeled data to a data pool. As another example, step 306 may include updating one or more of the classifiers without retraining. For instance, a neural network classifier may be updated via incremental learning processes.

In some examples, process 300 may include step 308, which may include determining if the uncertainty score meets a second uncertainty condition. For example, the second uncertainty condition may comprise a test of whether the uncertainty score is sufficiently low (the inferred pain score is sufficiently certain). For instance, step 308 may include determining a sample has an uncertainty value Q≤τ, where τ represents an uncertainty threshold. For example, in an example where Q is an entropy value, τ may be a number between, for instance, 0.1-2 (e.g., between 0.2-1, 0.2-0.6, 0.5, 0.6, etc.). In further examples, any suitable condition may be used that allows the selection of the relatively most certain examples for which the model is sufficiently confident.

While described with respect to entropy-based sampling, steps 305 and 308 may be performed via any suitable sampling method, such as uncertainty-based sampling, committee-based algorithms, and information density sampling methods, etc.

In some examples, process 300 may include steps 309 and 310, which may be performed if the uncertainty score meets the second condition in step 308. For example, step 309 may include generating a generated label for the subject data (e.g., sample corresponding to the uncertainty score). For instance, step 309 may include using prediction labels produced in step 303 as ground truth labels. Step 310 may include updating the sensed pain classifier of the pain assessment classifier based on the generated label. For example, step 210 may include updating a training dataset with the sample and generated ground truth label, and then updating one or both classifiers. As discussed with respect to step 306, in some cases, steps 308-310 may be performed with respect to a batch of samples. In these examples, step 310 may include updating a training data set with any samples (and generated ground truth labels) meeting the second condition.

In some examples, process 300 (e.g., steps 307, 310) may further include updating the first or second uncertainty conditions. For example, in some cases, the second uncertainty condition may be relaxed as more data samples are acquired in the training data set. For example, an initial value of τ may be established to select examples with relatively low Q. In this example, process 300 may include incrementally increasing τ, such as by adding a small Δ value with an interval I. Accordingly, in this example, as the model has access to more annotated data, it may become more reliable, supporting a larger value of Q for automatic label generation. For instance, in some cases, at the beginning of classifier development, there may be limited annotated data. In such cases, earlier models tend to be more noisy or less reliable (e.g., more uncertain) than later models. In some implementations, a maximum τ may be set. For instance, models trained with relatively large amounts of data may not be reliable in some cases due to several reasons (e.g., faulty acquisition sensors). A maximum possible value for τ (τₘₐₓ) may reduce a risk of selecting examples with low-quality prediction labels from the most recently trained pain assessment classifier.

In some examples, process 300 may further include step 311, which may include outputting the inferred pain score for the subject. For instance, step 311 may comprise outputting the inferred pain score to the subject or other user via a GUI, uploading the inferred pain score to a server, storing the inferred pain score in a database, etc. In some examples, step 311 may comprise outputting further pain-related information. For instance, step 311 may comprise generating a future pain prediction (e.g., based on past inferred pain scores), a possible pain condition diagnosis (e.g., based on a certain set of motions inducing higher levels of pain), an indication of success of ongoing rehabilitation (e.g., an effectiveness of ongoing physical therapy), etc. As another example, step 311 may comprise outputting information based on the inferred pain score. For instance, physiological data may be sent to a health provider based on a threshold pain score, etc.

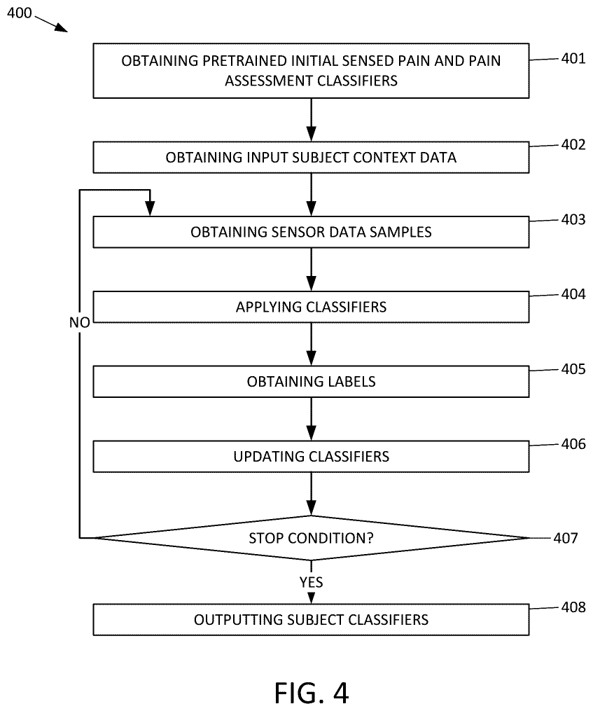

FIG. 4 illustrates an example process 400 for generating pain assessment classifiers. For example, process 400 may be used to generate models (e.g., pain classifiers) that are customized to a particular subject. In other examples, process 400 may be applied to generate models that are customized to a group of subjects (e.g., a sports team, a group of soldiers, etc.), a class/demographic group of subjects (e.g., neonates, children, male subjects, female subjects, elderly subjects, etc.) For example, process 400 may enable learning individualized differences in soldier pain during training and real-time combat. This personalization has the potential to improve soldiers' team performance through a more personalized assessment of each team member, allowing for future predictions of performance.

In some examples, process 400 may include step 401, which may include obtaining a pretrained sensed pain classifier and a pretrained pain assessment classifier. For example, the pretrained classifiers may be classifiers trained on an initial seed dataset of known users, labeled data samples, etc. As another example, step 401 may include obtaining pain classifiers that were generated during a previous performance of process 400 (e.g., subject A's customized classifiers may be used as initial classifiers for subject B). As another example, step 401 may include obtaining pretrained classifiers that were trained using subject data from the same subject (e.g., past subject data, data obtained during an initial model training session, etc.). In some implementations, generic pretrained classifiers may be used in process 400. In other implementations, pretrained classifiers may have characteristics related to the subject. For instance, a pain assessment system may include different pretrained classifiers for different populations, groups, etc. As discussed below, classifiers may be retrained and updated via an iterative process. In such examples, the pretrained classifiers may serve as the initial classifiers for a first iteration.

In some implementations, step 401 may comprise obtaining pretrained classifiers that correspond to a pain assessment sensing modality. For example, a pain assessment application to be instantiated on a smartwatch may apply to certain sensing modalities made available by the smartwatch (e.g., IMU data, heartrate data, etc.) In this example, the pretrained classifiers may be specific to such sensing modalities (e.g., the pretrained classifiers may be trained on training data obtained via similar smartwatches). As another example, a sports pain assessment system for a baseball pitcher be based on body sensors positioned on the subject's shoulders, arms, back, etc. In this example, step 401 may comprise obtaining classifiers trained on similar data. As a further example, operations on sensor data may be performed to extract certain features (e.g., velocity/energy associated to postural movements) while others may be performed to extract other features (e.g., static posture). In this example, step 401 may include obtaining pretrained classifiers trained using those sensor data.

In some examples, process 400 may include step 402, which may include obtaining input subject context data. For example, the input subject context data may be known context data related to the subject (e.g., health conditions, demographic information, survey answers, etc.). Process 400 may further include step 403, which may comprise receiving subject sensor data samples. For instance, step 403 may comprise receiving sensor data while a subject performs various movements or activities and tabulating the data into samples.

In some examples, process 400 may include step 404, which may comprise applying the current iteration's classifiers to the subject data. For example, step 404 may comprise applying the current iteration's sensed pain classifier to the sensor data samples obtained in step 403. As a further example, step 404 may comprise applying the current iteration's assessed pain classifier to the sensor data samples (from step 403), input context data (from step 402), and encoded unknown context (from the sensed pain classifier).

In some examples, process 400 may include step 405, which may include sampling outputs of step 404 to generate labels for additional training data (e.g., training data that is specific to the subject). For example, step 405 may comprise calculating uncertainty scores for the data samples based on the outputs of step 404. For instance, step 405 may comprise calculating combined entropy values for a sensed pain classifier and a pain assessment classifier (e.g., as discussed with respect to eq. (1) via other sampling techniques discussed herein). Step 405 may include generating a first set (e.g., one or more) of labels automatically based on a low uncertainty condition (e.g., an uncertainty score being less than or equal to a threshold t). For instance, the output(s) of the assessed pain classifier and/or the sensed pain classifiers may be appending to the data samples as ground truth values.

As another example, step 405 may include generating a second set of labels via manual labeling. For instance, manual labels may be requested for set number of samples (e.g., k), for samples meeting a high uncertainty condition, etc. For instance, step 405 may comprise requesting and receiving labels for the corresponding samples from a subject or other user via a GUI. For instance, requesting a set number of labels may provide a subject/user with a sense of agency or trustworthiness of the model given their involvement in the cooperative learning technology.

In some examples, process 400 may include step 406, which may include updating the classifiers based on the labels obtained in step 405. For instance, step 406 may comprise appending the labeled data samples (e.g., the labeled subset of samples received in step 403) to the training dataset used to train the initial classifiers obtained in step 401. As another example, in a second or later iteration, step 406 may comprise appending the labeled data samples to the training dataset used to train the current iteration's classifier. Step 406 may further comprise retraining the classifiers using the updated training dataset. For example, step 406 may comprise retraining the classifiers based on the entire updated training dataset. As another example, step 406 may comprise sampling the updated training dataset and retraining the classifiers based on the sampled training dataset. In further examples, any other suitable technique may be applied to use the newly labeled data to update the classifiers. For instance, the newly labeled data may be used to provide an incremental update to a neural network.

In some examples, process 400 may include step 407, which may include determining if a stop condition has been met for generating the classifiers. For example, step 407 may include evaluating the accuracy or other goodness of fit measure of the newly updated classifier and comparing the output to a threshold condition. As another example, step 407 may include determining if a threshold number of samples has been obtained, if a threshold number of labeled samples has been obtained, etc. As a further example, step 407 may include determine if a user has ended the classifier training process.

If the stop condition is not met, then process 400 may proceed to a next iteration. In some examples, various parameters may be updated for the next iteration. For example, as indicated above, a low uncertainty threshold may be increased as additional data is obtained (e.g., τ may be incremented by an increment Δ), the number of high uncertain samples may be reduced (e.g., k may be reduced), a number of samples to be obtained in a next iteration (e.g., the number of samples obtained in a next iteration of step 403), etc.

If the stop condition is met, then process may proceed to step 408, which may include outputting the latest iteration classifiers. For example, step 408 may include storing the classifiers in a patient's electronic medial records (ERMs), instantiating the classifiers in a pain assessment device (e.g., wearable device, smartphone, clinical device, etc.), personal medial device, home setting, server, etc. For instance, a patient may have instances of their classifiers stored in an ERM system, which may be instantiated on a computing device during a pain assessment session. In some examples, a subject may have multiple associated classifiers. For example, a record of past classifiers may be maintained (e.g., to provide the user information regarding the change of their model over time, highlight aspects of the cooperative learning process, etc.) As another example, the output classifiers from step 408 may be stored as candidates to replace existing classifiers. For instance, in situations where drug seeking may be an issue, the candidate classifiers may be stored until a physician or other supervising party approves the update.

### EXAMPLES AND EXPERIMENTS

Algorithm 1, described below, provides an example of processes and procedures that a pain assessment system may execute to generate a sensed pain classifier (referred to as a unknown context encoder or EUC) and a pain assessment classifier (referred to as a pain assessment model or PAM). As will be clear by context, in some instances, both the EUC and PAM will be referred to jointly as “PAM”

Algorithm 1: Personalized and context-
aware PAM using cooperative learning.

```
Input: Dₛ, Dₚₒₒₗ, Dₕₒ, λ, k, τ, τₘₐₓ, Δ, I
Result: Pain assessment model (PAM and/or EUC)
Procedure: trainPAM(Dₛ)
  EUC ← argminLearner_{euc}(Dₛ)
  PAM ← argminLearnerₚₐₘ(EUC, Dₛ)
  return EUC, PAM
i = 0
while constraint(s) do
  if S_{new} then
    Load most recently trained EUC, PAM
  end
  else
    EUC, PAM ← trainPAM(Dₛ)
  end
  Pₕₒ ← PAM(EUC(Dₕₒ, Dₕₒ)
  Pₚₒₒₗ ← PAM(EUC(Dₚₒₒₗ, Dₚₒₒₗ)
  Dₚₒₒₗ ← Rank(Dₚₒₒₗ[Q(Pₚₒₒₗ, λ)])
  Dₕₐ ← Dₚₒₒₗ[: k]
  Dₘₐ ← Dₚₒₒₗ[Q(Pₚₒₒₗ, λ)≤min(τ,τₘₐₓ)]
  if i%I == 0 then
    τ = τ + Δ
  end
  Ds ←Dₛ∪Dₕₐ∪Dₘₐ
  Dₚₒₒₗ ← Dₚₒₒₗ\(Dₕₐ∪Dₘₐ)
  i = i + 1
end
return EUC, PAM
```

In the proposed cooperative learning (COOL) framework (Algorithm 1), PAM is iteratively updated while constraints are met (line 9). In implementations, any suitable constraint may be applied, which may depend on a particular application or a specific scenario. For instance, example constraints include: i) |Dₚₒₒₗ|>0 (e.g., iterate as long as examples are present in Dₚₒₒₗ); ii) Q>predefined threshold (e.g., iterate while uncertainty scores exceed a predefined threshold); iii) accuracy<predefine threshold (e.g., iteration while the pain prediction are under a predefined threshold), etc. In lines 10-15 PAM is trained using a small seed dataset Dₛ collected from n users; Dₛ is a labeled dataset with contextual and sensing data. The case of lines 10-11 is discussed below with respect to a cold-start scenario. Then, in line 16 the algorithm tests and measures the performance of the loaded model on a held-out dataset Dₕₒ.

In line 17, P is inferred using PAM on a pool of unlabeled data Dₚₒₒₗ. Line 18 includes quantifying the quality of the prediction made by the PAM on Dₚₒₒₗ using Eqn. 1 and ranking the examples in Dₚₒₒₗ from most uncertain to certain (high Q indicates high uncertainty) by sorting examples in descending order using the Q associated with each example. Line 19 includes querying the top k examples (e.g., most uncertain) from the ranked Dₚₒₒₗ, and sending them for manual labeling (e.g., annotations by a user or other human) for feedback.

Line 20 includes selecting the examples with Q<τ, where τ represents the uncertainty or entropy threshold. This allows the selection of the relatively most certain examples for which the model is sufficiently confident. For these examples, prediction labels produced from the trained model PAM are retrieved and treated those labels as the ground truth labels. Note that at the beginning of the model development, such as in a situation with a low amount of prior annotated data, earlier models are likely to be noisier and less reliable. In this example, in lines 20-25, the algorithm applies a stricter constraint, which is a smaller value for τ to select examples with very low Q (e.g., 0.2, 0.3, 0.6, etc.). In lines 21-23, τ is incrementally increased by adding a small Δ value (e.g., a value around an order of magnitude smaller than the initial τ, 0.002, 0.003, 0.006, 0.001, 0.01, etc.) with an interval I. Here, the increasing τ may support the model having access to more annotated data and becoming more reliable. In some cases, there may be a limit on the maximum possible value for τ (τₘₐₓ). For example, this may reduce the risk of selecting examples with low-quality prediction labels from the most recently trained PAM, such as resulting from models trained with relatively large amounts of unreliable data (e.g., faulty acquisition sensors). For example, τₘₐₓ may be a value such as 1, 2, 3, 0.9, etc.

In line 24 annotations are obtained from the k most uncertain examples (Dₕₐ) from humans, and then Dₘₐ is obtained with pseudo-annotations (e.g., predicted labels) from the most recently trained PAM where Q(Dₘₐ)≤min (τ, τmax). Then, Dₕₐ and Dₘₐ are combined with Ds as shown in Algorithm 1. In line 25, the samples Dₕₐ and Dₘₐ are removed from the pool. In the next iteration, the augmented Ds is used to train the next version of the PAM. This process continues based in the specified constraints or other end conditions such as availability of resources like time and human annotators, or the number of examples in the dataset.

Additionally, specific performance targets for the model in terms of accuracy and model entropy may be used as discussed above. For the purposes examples described below, a limited number of examples available in the dataset is used for training. The algorithm is run until all the examples available in the dataset have been used. Specifically, the algorithm iterates while the number of examples in the dataset is greater than zero (e.g., |Dpool|>0; see Algorithm 1).

In the case of the cold start scenario (S_{new}), the framework starts with a PAM which was previously developed and deployed with known users. In other words, instead of starting with Dₛ to train PAM from scratch by collecting new data, the trained PAM is used to make inference P on new users' data. Then, Q is estimated based on P to trigger the collection of new annotated examples by the collaboration between S_{new} and PAM in an incremental fashion (Algorithm 1). The rest of the process is the same as the approach described above for non-new users.

### Evaluation

The PAM framework was evaluated using the EmoPain challenge dataset provided by the University College of London. This dataset was selected as publicly available pain dataset that has 1) known contextual information (e.g., CLB pain status) that cannot be predicted directly by the ML model, 2) unknown contextual information (e.g., physical activities) that can be encoded or predicted using the ML model, and 3) ground truth pain level labels. Along with the healthy control subjects, this dataset contains patients diagnosed with CLB pain for at least six months and were undergoing treatment; the patients were substantially disabled by CLB pain. Gender wise, approximately 1 of the subjects are male and the rest are female, and all subjects belong to the Caucasian and Asian ethnicity with an average age of 55.5 years.

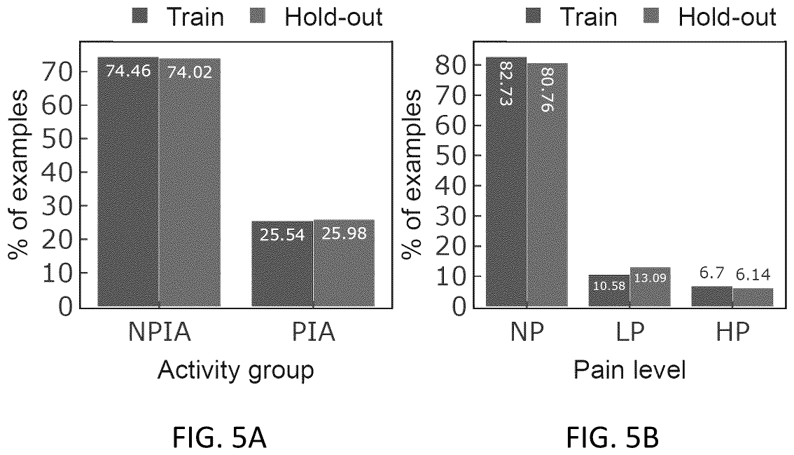

The dataset was collected using multiple devices including cameras, a custom motion capture suit (12 different sensors placed on different body locations), and electromyography (EMG) adhesive probes. The inertial measurement units (IMUs) in the suit captured the 3D Euler angle at 60 Hz while the EMG probes captured the lower and upper back muscles at 1,000 Hz. In evaluations described below, the IMU data was down sampled to 6 Hz and EMG data to 100 Hz by computing the moving average to speed up the experimentation. During data collection, medical professionals asked the subjects to perform two groups of physical activities: 1) pain-inducing activities (PIA): bending, one-leg-stand, reach-forward, sit-to-stand, stand-to-sit; 2) no-pain-inducing activities (NPIA): sitting still, standing still, walking, and others (e.g., self-preparation). FIGS. 5A and 5B show the percentage of examples in each activity group and each pain level observed in the EmoPain challenge dataset with data split for personalized model evaluation. Table 1 presents a brief summary of the dataset.

TABLE 1
Brief summary of EmoPain challenge data set

| | |
|---|---|
| Demography | females & males; Mean age: 55.5 years old |
| Population Type | Healthy control group & CLB patient |
| Data recording | Cameras (58fps), IMUs (60 Hz), EMG sensors |
| apparatus | (1,000 Hz) |
| Sensing modalities | Angle (13), energy (13), EMG (4) |
| Physical activities | PIA: bending, one-leg-stand, reach-forward, |
| group | sit-to-stand, stand-to-sit |
| | NPIA: sitting still, standing still, walking, |
| | and others (e.g., self-preparation) |
| Pain levels | NP, LP, HP |

### Sensing and Contextual Modalities

IMU sensors were used to collect the postural information from different anatomical joints (flexion, knee, elbow, shoulder, lateral bend, and neck). EMG probes were used to capture the muscle movement information from the lower and upper back muscles. The body posture information was captured, by computing the angular information (angle) in 3D space. This is performed to collect anatomical movement of the body (i.e., postural information), which is represented by a feature vector of size 13. The angular velocity (energy), which was computed from the postural information over time, captures the range of motion of the patients. The angle data is then used to compute the energy, which represents the angular velocity of the body movement using a feature vector of size 13. Muscle activity levels were captured using EMG probes from the right and left lumbar paraspinal (lower back) and right and left upper Trapezius (upper back) muscles, which is represented by a feature vector of size 4.

In addition to the sensing modalities, the dataset has known context modality and unknown context modality. The known context modality is the CLB status, which is represented as a binary variable indicating whether a given user is a CLB patient or not. The unknown context is the group of activities such as the PIA group and NPIA group that were performed by the subjects.

### Data Split for Personalized Evaluation

For personalized evaluation, the dataset was split into two different portions: training and hold-out test set. More than half of the subjects in the EmoPain Challenge dataset participated in two different data recording sessions while the rest participated in only one recording session. Those who participated in only one session were included in the training; for those who participated in two sessions, one session was randomly selected from each subject to include in the training set, and the other session was included in the hold-out test set. The observed pain level during the experiments was annotated as no pain (NP), low-level pain (LP), and high-level pain (HP). The distribution of the different pain classes and activity groups is highlighted in FIGS. 5A and 5B which illustrates high-class imbalance. To compare with the SOTA, the framework was also evaluated using the original hold-out (subject-independent) set provided in the dataset. The obtained results are presented below.

### Implementation Details for Evaluation

The cases discussed below were implemented with random forests (RF) as the learning algorithm for both EUC and PAM. For example, RFs may be applicable where i) the studied wearable data is tabular data (e.g., tree-based models are useful for tabular data); ii) RF is computationally fast; iii) RF provides out-of-bag estimation; iv) RF may provide better generalization and require less parameter tuning than other approaches; v) RF is reliable in cases of scarce and skewed (outliers) training examples; these characteristics make RF useful for the rapid updating of the assessment models. EUC was trained as a binary classifier to compute the probabilities of PIA and NPIA, which was combined with the sensing modalities (e.g., energy, angle, EMG), and context modality (e.g., CLB status) to train PAM. The tree estimator parameter was empirically selected to be 750. 20% of the examples were sampled stratifying over subjects from the entire training set in which 1% of examples were used as the seed (small labeled) dataset Ds, and 19% of the examples were used as unlabeled pool dataset Dₚₒₒₗ. Dₛ set was sampled 5 times with 5 different random seeds for each experiment and results were obtained on a hold-out test set over 5 seeds. The performance of PAM was evaluated using two evaluation metrics: accuracy and MCC (Matthews correlation coefficient). MCC has a range of [−1, 1], where a value closer to −1 indicates poor performance while a value closer to 1 indicates good performance. Other measurements for a subset of the results include class-specific precision, recall, and f1-score.

### Quantifying the Contribution of Modalities

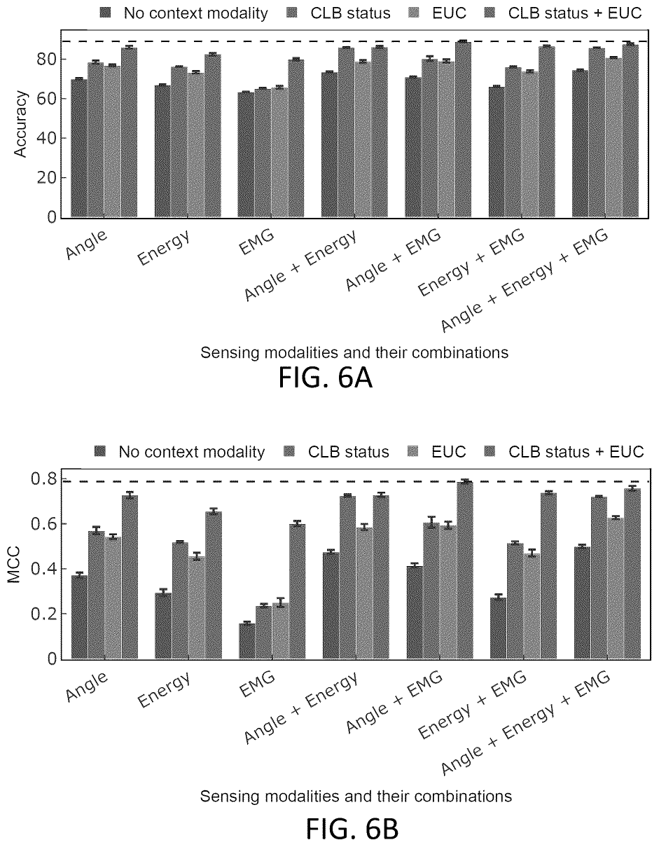

An ablation study was performed on the sensing modalities: angle, energy, EMG, and on the contextual modalities: CLB status, and EUC. To quantify the importance of each pain modality and their combination in this example, an exhaustive search over all possible combinations of context and sensing modalities was performed, which is highlighted in FIGS. 6A (accuracy) and 6B (MCC). For example, the first bar in both plots of FIG. 3 shows the results when we only used angle sensing modality while the last bar shows the combination of all three sensing modalities (angle, energy, EMG), and context modalities (CLB status, and EUC). This is performed to quantify the impact of each modality and identify the best combinations for pain assessment.

FIGS. 6A and 6B illustrate that the lowest performance (in terms of accuracy and MCC) was obtained when context modalities were not used. Also, performance improved for each possible combination of the sensing modalities with the inclusion of each context modality and their combination. In this example, Δ_{metric} is defined as the difference in performance between two models measured via a given metric, e.g., Δ_{accuracy}=Accuracy_{Modelj}−Accuracy_{Modeli}; where Accuracy_{Modelj}, and Accuracy_{Modeli} are accuracy obtained from Modelⱼ, and Modelᵢ, respectively. Specifically, comparing the results in FIGS. 6A and 6B against the best performing PAM (trained with the sensing modalities only) using Δ_{accuracy}, and Δ_{MCC} illustrates that i) combining CLB status with the sensing modalities leads to maximum improvement of Δ_{accuracy}=11.49, and Δ_{mcc}=22.6 points; ii) combining EUC with the sensing modalities leads to maximum improvement of Δ_{accuracy}=6.38, and Δ_{mcc}=12.84 points; iii) combining both CLB status and EUC with the sensing modalities leads to maximum improvement of Δ_{accuracy}=14.57, and Δ_{mcc}=28.8 points. These results show that context (e.g., CLB status, EUC) may improve the performance of PAM, especially when combining multiple context modalities with sensing modalities.

In this example, the best performing PAM was obtained with the combination of CLB status, EUC, angle, and EMG−accuracy=89±0.5% and MCC=0.787±0.01. As this specific combination achieved the best performance of PAM, it was used for pain assessment in the rest of the experiments.

### Experimental Results

In this experiment, the experimental setup was as described above. The parameters for the method were set as follows: equal weights (λ=0.5) to both EUC and PAM; for human annotation, k=10 examples in each iteration; for annotation from the most recent PAM, τ=0.1, I=10, Δ=0.01, τmax=0.4; τ was set to 0.1 to select examples for which PAM is most likely certain, and the model waited for I=10 training iteration before increasing τ by adding Δ=0.01. A small value of Δ=0.01, and τₘₐₓ=0.4 were set to avoid selecting and using noisy pseudo-labels. Ablation studies on the impact of these parameters were performed an are discussed below. In terms of constraint, the COOL iteration was repeated until all available examples in Dₚₒₒₗ were exhausted. The results of this experiment are reported in Table 2.

TABLE 2
Experimental results

| Categories | Precision | Recall | F1-score | Accuracy (%) | MCC |
|---|---|---|---|---|---|
| NP | 0.8742 ± 0.0155 | 0.98510 ± 0.0083 | 0.9262 ± 0.0084 | 87.71 ± 1.09 | 0.7620 ± 0.0215 |
| LP | 0.9106 ± 0.0340 | 0.7787 ± 0.00518 | 0.8379 ± 0.0234 | | |
| HP | 0.8150 ± 0.0658 | 0.50270 ± 0.1022 | 0.6128 ± 0.0671 | | |

The proposed COOL framework achieved competitive performance (accuracy=87.71±1.09% and MCC=0.7620±0.0215) as compared to the results obtained above (accuracy=89±0.5%; MCC=0.787±0.01). Notably, with respect to FIGS. 6A and 6D, human-annotated labels were used for all examples. However, here, only a subset of the human-annotated examples was used (<8% of what was used above) while the rest were labeled by the model. As for class-specific performance, the best result was obtained for NP with relatively low performance for LP and HP. This relatively poor performance for LP and HP could be attributed to the class imbalance issue highlighted in FIGS. 5A and 5B.

### Simulated Cold Start Scenario

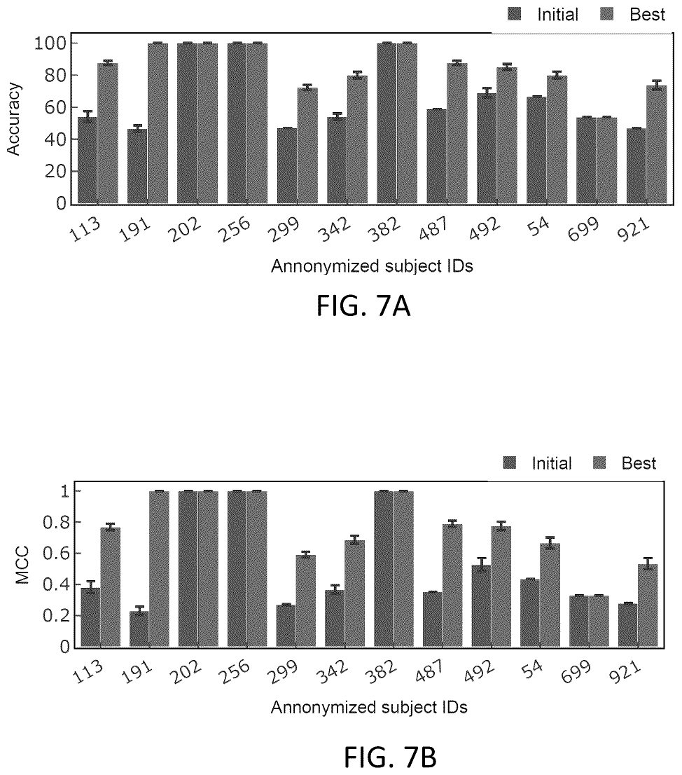

As discussed above, in a cold start scenario, instead of starting from scratch by collecting a seed dataset Ds for new users, the most recently developed PAM that was trained on known users is used as a prior. To simulate the cold start scenario, the subjects who participated in only one data collection session were treated as known users. Then, the seed PAM was trained using this dataset, and parameters λ, k, τ, I, τₘₐₓ, and Δ similar to what was set above with respect to the results section. Once the seed model was obtained, COOL was performed on each subject separately to quantify the performance of PAM on each new user. In other words, for a new user S_{new}, the session of a specific user that exists in the training set was treated as a Dₚₒₒₗ while the other session of the same subject (S_{new}) was treated as the hold-out set used to determine the model performance. This simulation was performed for each subject who has at least two data recording sessions. FIGS. 7A and 7B illustrate the simulation of a cold start scenario with new users. FIGS. 7A and 7B illustrate performance obtained with the ‘initial’ model, and the ‘best’ performing model using COOL. As illustrated, model performance was boosted with the incorporation of the new examples from the target subjects, using COOL framework.

As illustrated, the performance of the initial PAM was suboptimal when it was used to make inferences on each S_{new}. However, with further training with the data from the S_{new} using COOL, the performance of PAM drastically improved in most cases. Interestingly, it was observed that for some subjects (such as 202, 256, 382), the initial PAM model achieved good performance. This suggests the benefit of using the ‘Initial’ model along with the ‘Best’ model when dealing with unknown or new users (e.g., a cold start scenario). Finally, the aggregated performance of all new users for both the ‘Initial’ and ‘Best’ models was measured. The ‘Initial’ model achieved an accuracy of 67±21% and MCC of 0.52±0.30 across users while the ‘Best’ model achieved an accuracy of 86±14% and MCC of 0.77±0.21; an improvement of Δ_{accuracy}=19 and Δ_{mcc}=25 points. This improvement shows the positive impact of personalization on performance.

To assess the significance of the improvement, a one-sided t-test was performed for paired samples between the ‘initial’ and ‘best’ models. The alternative hypothesis (H1) is that the performance of ‘best’ is significantly (α=0.05) better than ‘initial’ due to the incorporation of new examples using COOL from S_{new}. The test statistics were separately measured for accuracy and MCC, obtaining test statistics of 8.8219 with p-value<0.001, and 8.5726 with p-value<0.001, respectively. These results provide evidence that the incorporation of new examples from S_{new} significantly contributes toward the improvement of PAM.

### Ablation Studies

Various ablation studies were performed to explore the impact of various parameter values, in the experimental set-up discussed above (e.g., with the EmoPain dataset and described features, etc.). These results may present various considerations to be applied when deploying the proposed technology in particular implementations.

### Ablation Study—λ

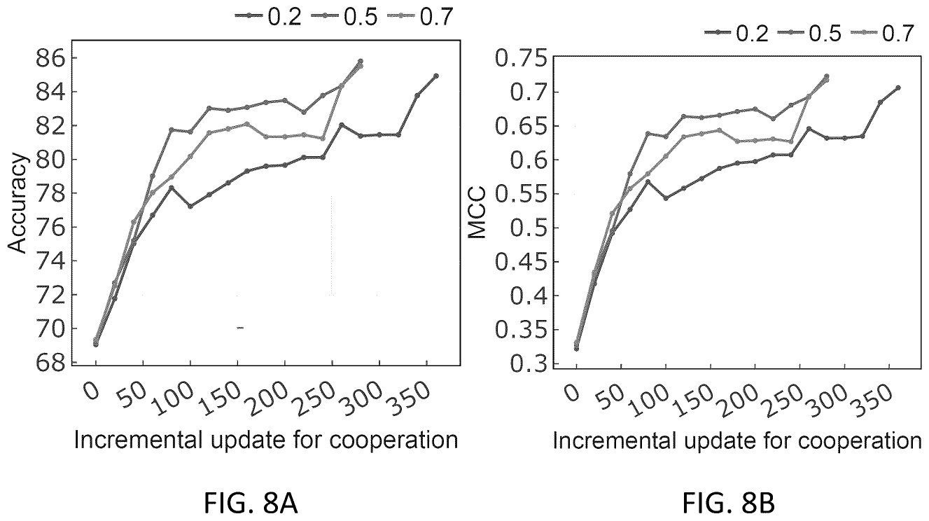

Some use cases may weigh PAM higher than EUC and vice versa. An ablation study was performed on the weighting parameter λ. To evaluate the impact of λ, the following set of values {0.2, 0.5, 0.7} of λ in Eqn. 1 were employed. The rest of the parameters for COOL were as follows: T=0.1, I=2, Δ=0.01, τₘₐₓ=0.4, and k=5. The obtained results are shown in FIGS. 8A (accuracy) and 8B (MCC). In this example, equally weighted EUC and PAM led to the best performance (λ=0.5). Additionally, giving less weight to PAM (λ=0.2) required more incremental updates while giving more weight to PAM (λ=0.7) did not lead to any improvement in the performance, in this example. This result indicates the benefit from contextualization in the PAM.

### Ablation Study—k

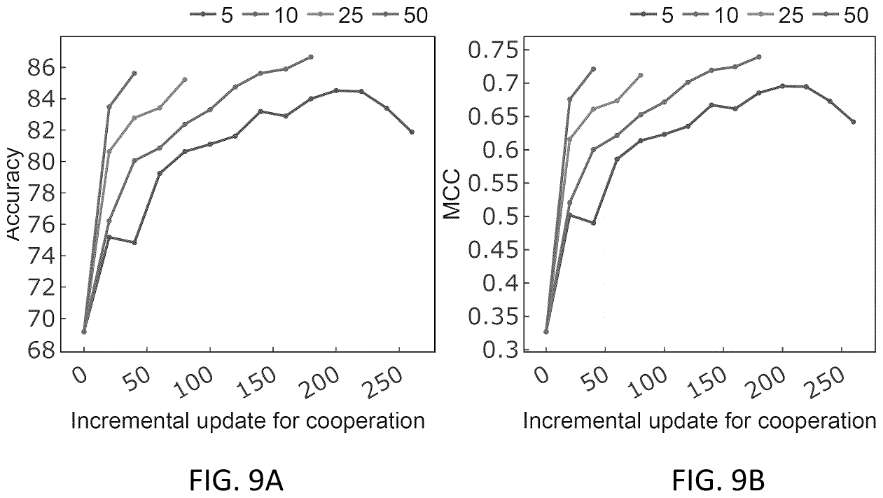

As discussed above, how often the cooperative human (e.g., subject, practitioner, or other user) annotates examples for model development may impact user experience and model quality. An ablation study was performed to evaluate potential values of k, with the following set of k values {5, 10, 25, 50} (e.g., k=5 means the user needs to annotate 5 examples in each iteration of the COOL algorithm). The rest of the parameters were set as follows: τ=0.1, |=2, Δ=0.01, τₘₐₓ=0.4, λ=0.5. The results are illustrated in FIGS. 9A (accuracy) and 9B (MCC). In this example, a higher k led to a smaller number of incremental updates but user experience may be impacted by a higher k. In this example, k=10 provided a steep improvement in the performance (accuracy, MCC) while requiring a relatively smaller number of updates as compared to when k=5. Also, from a user experience standpoint, k=10 may be useful as, for each example, the user answers two questions: the pain level and the type of activities performed. Hence, annotating 10 examples may provide an acceptable user experience. In the case of k=5, the degradation in this example could be due to poor PAM annotated examples obtained from prior PAM as it increased the τ with more iterations.

### Ablation Study—I

As discussed above, in some examples, a certainty threshold for automatic label generation may be increased as model updates are performed. An ablation study was performed to explore how often it would be useful to increase the τ by adding 0.01 (Δ). In this example, initially τ=0.1 and τₘₐₓ=0.4. Then, the following set of values for I were used: {2, 5, 10, 25, 50}, where smaller values (e.g., I=2) indicate a rapid update of τ and a selection of high entropy examples over the iterations while larger values (e.g., I=50) indicate otherwise. Other parameters were set as λ=0.5 and k=5 and COOL was performed until all available examples in Dₚₒₒₗ are exhausted. An example goal of I is to provide high accuracy and MCC while reducing the number of incremental updates.

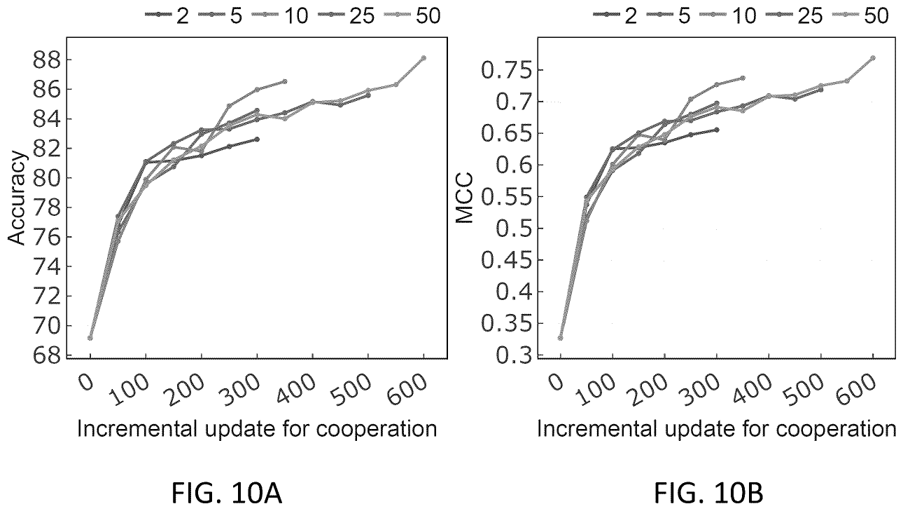

FIGS. 10A (accuracy) and 10B (MCC) illustrate the results of this experiment. In this example, I=10 represented the best value as it achieved competitive performance with a relatively small number of updates. It queried 350 times for human and machine annotation of new examples. Another result illustrated is the steep increase in performance and that when τ is updated with a larger interval I, it requires more incremental updates. This may correspond to relatively more human intervention and computational resources. Conversely, I<10 led to lower accuracy and MCC while requiring competitive incremental updates per iteration for cooperation.

### Ablation Study—Initial Size of Dₛ

As discussed above, PAM models may be initially trained with an annotated data set, such as from a labeled seed dataset obtained from a pool of users or from a seed data set obtained from a single user. To explore how many human-labeled examples are needed for training the initial seed PAM, the following percentages of the EmoPain dataset were analyzed as the size of the initial Dₛ: {0.2%, 0.5%, 1%, 2%}. For instance, 1% means only 1% of the original training dataset was used for developing the initial PAM. The Dₚₒₒₗ was then queried for new examples using the COOL. In this example, the rest of the parameters were set as follows: T=0.1, I=2, Δ=0.01, τₘₐₓ=0.4, λ=0.5, I=10.

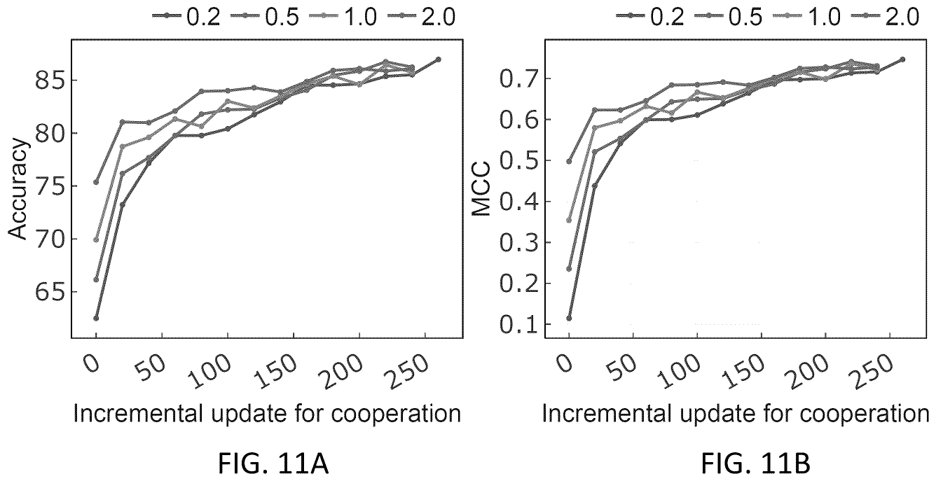

FIGS. 11A (accuracy) and 11B (MCC) illustrate results of this experiment. As illustrated, with a similar number of COOL updates, the PAM performance is similar, particularly when the number of updates is >150. However, with a relatively smaller Dₛ, earlier in the model development, the performance (accuracy, MCC) is relatively lower than a larger Dₛ size. For instance, when Dₛ size was 0.2%, initial MCC=0.1 while initial MCC=0.5 when Dₛ=2%. Accordingly, while using a relatively larger seed dataset size provides a head start, a smaller seed dataset size can eventually lead to a similar PAM performance with enough incremental updates (e.g., 0.2% took >250 updates).

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

What is claimed is:

1. A method, comprising:
obtaining subject data for a subject comprising sensor data and pain context data;
applying a sensed pain classifier to the sensor data to determine a sensed context;
applying a pain assessment classifier to the pain context data and the sensed context to determine an inferred pain score;
determining an uncertainty score based on the sensed context and the inferred pain score;
responsive to the uncertainty score meeting a first threshold condition:
requesting a manual label for the subject data;
updating the sensed pain classifier or the pain assessment classifier based on the manual label;
responsive to the uncertainty score meeting a second threshold condition:
generating a generated label for the subject data;
updating the sensed pain classifier or the pain assessment classifier based on the generated label; and
outputting the inferred pain score for the subject.

2. The method of claim 1, wherein the second threshold condition is based on a count of determined inferred pain scores for the subject.

3. The method of claim 1, further comprising:
responsive to the uncertainty score meeting a first threshold condition:
updating the sensed pain classifier and the pain assessment classifier based on the manual label; and
responsive to the uncertainty score meeting a second threshold condition:
updating the sensed pain classifier and the pain assessment classifier based on the generated label.

4. The method of claim 1, further comprising:
obtaining a batch of sensor data corresponding to a plurality of subject motion samples;
applying the sensed pain classifier to the batch of sensor data to determine a corresponding plurality of sensed contexts for the plurality of subject motion samples;
applying the pain assessment classifier to the pain context data and the plurality of sensed contexts to determine a corresponding plurality of inferred pain scores for the plurality of subject motion samples;
determining a corresponding plurality of uncertainty scores for the plurality of subject motion samples based on the plurality of sensed contexts and the plurality of inferred pain scores;
ranking the plurality of inferred pain scores based on the plurality of uncertainty scores; and
requesting a plurality of manual labels for k most uncertain inferred pain scores.

5. The method of claim 4, further comprising:
generating a generated label for any inferred pain scores having an uncertainty below a threshold value.

6. The method of claim 5, further comprising:
increasing the threshold value after determining a count/of inferred pain scores.

7. The method of claim 5, further comprising:
appending data samples corresponding to the manual labels and data samples corresponding to the generated labels to a seed dataset to generate an updated seed data set;
updating the sensed pain classifier using the updated seed data set by retraining the sensed pain classifier using the updated seed data set; and
updating the pain assessment classifier by retraining the pain assessment classifier using the updated seed data set and the updated sensed pain classifier.

8. The method of claim 1, wherein the uncertainty score comprises a linear combination of a sensed context entropy value and an inferred pain score entropy value.

9. The method of claim 1, wherein the sensor data comprises physiological data obtained from a wearable sensor.

10. The method of claim 9, wherein the sensor data comprises at least one of electromyograph data, posture angle data, and posture angular velocity data.

11. A pain assessment system, comprising:
a wearable sensor to obtain sensor data for a subject;
a context input to obtain pain context data for the subject;
a processor;
a non-transitory computer readable medium storing instructions executable by the processor to:
apply a sensed pain classifier to the sensor data to determine a sensed context;
apply a pain assessment classifier to the pain context data and the sensed context to determine an inferred pain score;
determine an uncertainty score based on the sensed context and the inferred pain score;
responsive to the uncertainty score meeting a first threshold condition:
request a manual label for the sensor data;
update the sensed pain classifier or the pain assessment classifier based on the manual label;
responsive to the uncertainty score meeting a second threshold condition:
generate a generated label for the sensor data;
update the sensed pain classifier or the pain assessment classifier based on the generated label; and
output the inferred pain score for the subject.

12. The system of claim 11, wherein the second threshold condition is based on a count of determined inferred pain scores for the subject.

13. The system of claim 11, wherein the instructions are further executable by the processor to:
responsive to the uncertainty score meeting a first threshold condition, update the sensed pain classifier and the pain assessment classifier based on the manual label; and
responsive to the uncertainty score meeting a second threshold condition, update the sensed pain classifier and the pain assessment classifier based on the generated label.

14. The system of claim 11, wherein the instructions are further executable by the processor to:
obtain a batch of sensor data corresponding to a plurality of subject motion samples;
apply the sensed pain classifier to the batch of sensor data to determine a corresponding plurality of sensed contexts for the plurality of subject motion samples;
apply the pain assessment classifier to the pain context data and the plurality of sensed contexts to determine a corresponding plurality of inferred pain scores for the plurality of subject motion samples;
determine a corresponding plurality of uncertainty scores for the plurality of subject motion samples based on the plurality of sensed contexts and the plurality of inferred pain scores;
rank the plurality of inferred pain scores based on the plurality of uncertainty scores; and
request a plurality of manual labels for k most uncertain inferred pain scores.

15. The system of claim 14, wherein the instructions are further executable by the processor to:
generate a generated label for any inferred pain scores having an uncertainty below a threshold value.

16. The system of claim 15, wherein the instructions are further executable by the processor to:
increase the threshold value after determining a count/of inferred pain scores.

17. The system of claim 16, wherein the instructions are further executable by the processor to:
append data samples corresponding to the manual labels and data samples corresponding to the generated labels to a seed dataset to generate an updated seed data set;
update the sensed pain classifier using the updated seed data set by retraining the sensed pain classifier using the updated seed data set; and
update the pain assessment classifier by retraining the pain assessment classifier using the updated seed data set and the updated sensed pain classifier.

18. The system of claim 11, wherein the uncertainty score comprises a linear combination of a sensed context entropy value and an inferred pain score entropy value.

19. The system of claim 11, wherein the sensor data comprises physiological data obtained from a wearable sensor.

20. A non-transitory computer readable medium comprising instructions executable by a processor to:
apply a sensed pain classifier to sensor data to determine a sensed context;
apply a pain assessment classifier to pain context data and the sensed context to determine an inferred pain score;
determine an uncertainty score based on the sensed context and the inferred pain score;
responsive to the uncertainty score meeting a first threshold condition:
request a manual label for the sensor data;
update the sensed pain classifier or the pain assessment classifier based on the manual label;
responsive to the uncertainty score meeting a second threshold condition:
generate a generated label for the subject data;
update the sensed pain classifier or the pain assessment classifier based on the generated label; and
output the inferred pain score.
